(19) United States
(12) Patent Application Publication
(10) Pub. No.: US 2021/0057079 A1
(43) Pub. Date: Feb. 25, 2021

(54) SYSTEM AND METHOD FOR TEACHING ACTIONS TO DEVELOP INDIVIDUALIZED, FOCUSED DECISION-MAKING SKILLS OVER TIME

(71) Applicant: ZEN HEALTH TECHNOLOGIES, INC., Carlsbad, CA (US)

(72) Inventor: Sibyl Badugu, Carlsbad, CA (US)

(73) Assignee: ZEN HEALTH TECHNOLOGIES, INC., Carlsbad, CA (US)

(21) Appl. No.: 16/997,219

(22) Filed: Aug. 19, 2020

Related U.S. Application Data
(60) Provisional application No. 62/891,096, filed on Aug. 23, 2019.

(51) Int. Cl. G16H 20/70; G09B 5/12

(52) U.S. Cl. CPC G16H 20/70; G09B 5/12

(57) ABSTRACT

The system and method can be used to teach individuals to quickly and easily stop incessant, repetitive thinking. Because each person is unique and has developed their own ways to deal with internal and external stimuli, the system contains tailored approaches to address the typical ways in which the general population approaches the world. Choosing a goal (typical issue) allows for targeting exercises that are designed for individualized approaches. Exercises can typically be performed very quickly (usually around 30 seconds) and can usually be completed just about anywhere. A quick check-in before and after completing an exercise allows for a quick evaluation if performing exercise worked effectively. If exercises have been effective for the individual, they can be saved for quick retrieval.

### BACKGROUND

### Field of the Invention

The invention is directed to a system and method for making the human mind aware that it is in a constant state of flux and for improving cognitive state, such as decision-making. In some embodiments, the system and method can be configured to teach non-reactivity to external and internal stimuli using electronic devices such as a smart phone, tablet PC, or a web-browser presented via a computer-connected display screen.

### Description of the Related Art

U.S. Pat. No. 8,562,355, issued Oct. 22, 2013, titled “SYSTEM AND METHOD FOR GUIDING NEXT STEP ADAPTIVE BEHAVIOR”, discloses a system and method configured to process clarified emotional response data in order to guide next step adaptive behavior. Here, emotional responses with predetermined clarified intensities are sorted. The best quality clarified intensity or intensities are then selected as the ‘heart of the matter’, which is then shown to a user to reveal how the user is most uniquely responding to a particular user or issue. The user is then given guidance on how to respond to the particular user or issue thereby guiding the user's behavior in response to such user or issue.

U.S. Pat. No. 9,401,097, issued Jul. 26, 2016, titled “Method and apparatus for providing emotion expression service using emotion expression identifier”, discloses a method and apparatus for providing an emotion expression service using an emotion expression identifier that substantially obviates one or more problems. According to an embodiment, a method for providing an emotion expression service using an emotion expression identifier includes collecting emotion evaluations of users about content related to a word or a phrase, the emotion evaluations being performed by the users after the users view the content, and displaying an emotion expression identifier representing the collected emotion evaluations of the users in the vicinity of the word or the phrase. According to another embodiment, an apparatus for providing an emotion expression service using an emotion expression identifier includes a collector configured to collect emotion evaluations of users about content related to a word or a phrase, the emotion evaluations being performed by the users after the users view the content, and a display controller configured to display an emotion expression identifier representing the collected emotion evaluations of the users in the vicinity of the word or the phrase. According to yet another embodiment, a method for providing an emotion expression service using an emotion expression identifier includes collecting emotion evaluations of users about a specific place and displaying an emotion expression identifier representing the collected emotion evaluations of the users at a point corresponding to a geographical position of the specific place on a map.

U.S. Pat. App. Pub. No. 2017/0287354, published Oct. 5, 2017, titled “MENTAL STRENGTHENING & PROGRAMMING SYSTEMS & METHODS”, discloses systems and methods produced in modules to accelerate learning, creating & increasing ability and programming habits. The modules in order the user experiences are the module that teaches the predictable cycles of human consciousness and life to educate the User to understand what to do to improve their life and how to use the technology as effectively as possible to create maximum acceleration of increase of ability and programming of desired habits; a module that leads them to analyze themselves to determine what habits of ability and belief they will choose to improve; another module teaches and trains and then presents the techniques of mindfulness to accelerate the user developing the habit of identifying and releasing from the habit of ignoring life and neglecting their abilities to embrace nurturing their abilities to create functional habits of fulfilling their desires and needs to enjoy a life of their choosing, then there are three sets of three modules on the three modalities of thought, Symbols, Imagination & emotion and language, the first module educates and trains them to understand and execute each modality with effective skill, the second module is a library of Sets of content organized to program habit and create ability, the last module is a practice module that presents the content for the user to emulate and imitate causing the user to generate and control levels of energy beyond their ability without the presence of the content to guide their thoughts & emotion flow, this module will be adapted to accept content of any discipline of knowledge to enable the user to accelerate learning, then there is a module that operates in a mobile devise to trigger the User to continue generating and controlling the thoughts and emotions they desire to turn into new or improved ability or a new habit; last is the module that guides the user to plan, schedule and monitor their practice or programming sessions and other areas of their life to sustain consistent accelerated growth of ability and programming and refining of habits.

U.S. Pat. App. Pub. No. 2017/0365184, published Dec. 21, 2017, titled “SYSTEM TO RE-TRAIN ONE'S REACTION TO NEGATIVE INTERFERENCE”, discloses a method for having an individual determine attributes about themselves that are associated with them performing their best (creating their box). The individual then recognizes when something has taken them out of their box, where their performance may not be optimum or where they may act in a way that they would not normally. The individual then determines the emotions associated with these changes and what triggered these emotions. They may then take steps to get themselves back into their box, so that they are at their best.

The current state of the art involves identifying an upcoming situation or habit that needs to be prepared for. Specific guidance and actions are prescribed as a behavior response to that situation.

### SUMMARY

In one embodiment of the present invention, a smartphone, tablet PC, or personal computer, is adapted, for example, with a software application, collectively “software-enabled device”, to provide specific actions to a user in order to learn how to interrupt the automatic repetitive workings of the user's mind and to determine if the recommended action was effective for the individual user.

In another embodiment, repeated use of the software-enabled device is configured to track, analyze and/or illustrate to the user a measure of effectiveness and improvement of non-reactive decision-making over time.

In another embodiment, results from performing recommended actions are easily understood and provide motivation to continue to interrupt the mind to new and personalized methods of making focused decisions.

Other embodiments will be understood by one having skill in the art upon a thorough review of the instant disclosure and accompanying drawings.

### Problem

The human mind spends most of its time with continuous, silent conversations. Most of these thoughts tend to involve going over an event that has already occurred. Other times, these thoughts tend to involve future situations that are predicted to happen.

In between this constant thinking, decisions are made. Mostly small decisions that are needed during the day as to adhere to established routines. Other times, these decisions can be substantial (leading to long-term life decisions).

Decision-making skills are not formally taught in traditional educational institutions. If taught at all, they require a person to become aware that the decision-making skills currently used may need to be reviewed and revised. Oftentimes, the decision-making process tends to be reactive based on previous experiences.

This process of reactive, continuous decision-making caused symptoms in the body, mostly around the head (above the neck). Sometimes these symptoms show up as tension in the stomach, back or upper shoulder and neck area.

### Solution

A system and related method configured to consistently and repeatedly interrupt incessant thinking to allow creating of new individual methods of being centered and focused over time in order to learn to address internal and external stimuli non-reactively.

### BRIEF DESCRIPTION OF THE DRAWINGS

FIG. 1 shows a schematic representing a method for improving cognitive state in a user including several method steps for achieving the same.

FIG. 2 shows a graphical user interface (GUI), in accordance with one embodiment, for a software-application adapted to execute the method of FIG. 1.

FIG. 3 shows a GUI for a software-application where one goal can be selected from a plurality of possible goals associated with improving cognitive state of the user.

FIG. 4 shows a GUI for a software-application where mental-state indicators are displayed and selectable by the user for performing a self-assessment of mental state.

FIG. 5 shows an example of an exercise associated with a selected goal.

FIG. 6 shows another example of an exercise associated with the selected goal.

FIG. 7 shows yet another example of an exercise associated with the selected goal.

FIG. 8 shows a GUI prompting the user to repeat the method according to a second goal, or to finish the software-application.

FIG. 9 shows a GUI configured to display historical data for assessing improvement in cognitive state of the user over time.

### DETAILED DESCRIPTION

In one embodiment, a method for improving cognitive state in a user is disclosed, the method comprises: (i) executing a software-application configured to present a plurality of exercises to the user on a display screen; with the software-application: selecting a goal from a plurality of possible goals, wherein each of said goals is associated with a decision-making activity; and first-selecting one or more mental-state indicators to obtain an initial self-assessment of the mental state of the user; followed by: completing one or more exercises associated with the selected goal; and second-selecting one or more mental-state indicators to obtain a subsequent self-assessment of the mental state of the user.

In some embodiments, after obtaining the subsequent self-assessment of the mental state of the user, a second goal is selected, wherein the method is repeated according to the second goal.

In some embodiments, after obtaining the subsequent self-assessment of the mental state of the user, the method further comprises: evaluating historical data for assessing cognitive improvement.

In another embodiment, a system is disclosed, the system is adapted to execute, in connection with a non-tangible computer readable medium, a method for improving cognitive state in a user, wherein the method comprises: executing a software-application configured to present a plurality of exercises to the user on a display screen; with the software-application: selecting a goal from a plurality of possible goals, wherein each of said goals is associated with a decision-making activity; and first-selecting one or more mental-state indicators to obtain an initial self-assessment of the mental state of the user; followed by: completing one or more exercises associated with the selected goal; and second-selecting one or more mental-state indicators to obtain a subsequent self-assessment of the mental state of the user.

In some embodiments, the display screen may be coupled to a mobile phone.

In other embodiments, the display screen may be coupled to a personal computer, including: a tablet PC, laptop PC, or desktop PC.

In some embodiments, the non-tangible computer readable medium is stored on an internet network.

In other embodiments, the non-tangible computer readable medium is locally stored on memory associated with a device.

For purposes herein, the term “goal” means an objective related to improvement of cognitive state, such as, but not limited to: decision-making. Other examples of goals may include: establishing boundaries; staying focused; setting up routines; improving communication; getting organized; being flexible; and dealing with stress.

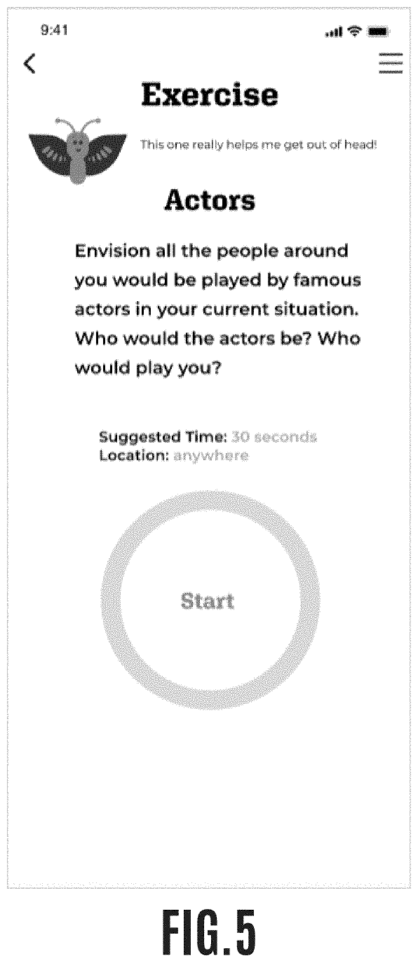

For purposes herein, the term “exercise” means an activity, associated with a selected goal, that is designed to stimulate a response in the mental state of the user for the purpose of improving cognitive state. While a myriad of exercises may be implemented in accordance with the knowledge and skill in the art, three explicit examples are illustrated in FIGS. 5-7 herein.

A goal can be selected prior to reviewing exercises associated with the selected goal. Each goal may be associated with a navigation expert (character animation) that is associated with the selected goal.

Once a goal is selected, one or more, and preferably three exercises are displayed for selection by the individual user.

Each exercise is associated with a suggested time required for completion. Initially, the suggested time may be short, for example, 30 seconds long—though the exercise may be performed for as long as the individual wants to engage the exercise.

An evaluation system is displayed for measuring the effectiveness of the exercise both before and after the exercise is performed.

Tracking the effectiveness of performing the exercises over time can be viewed and filtered by various metrics, such as date/time range.

Suggested use includes using the system and method a few times a day, initially to notice thinking patterns, then later, to learn to interrupt them and improve cognitive state, such as decision-making ability. It is expected that progressive use over time will show the measurable effectiveness.

Now, turning to the illustrated examples:

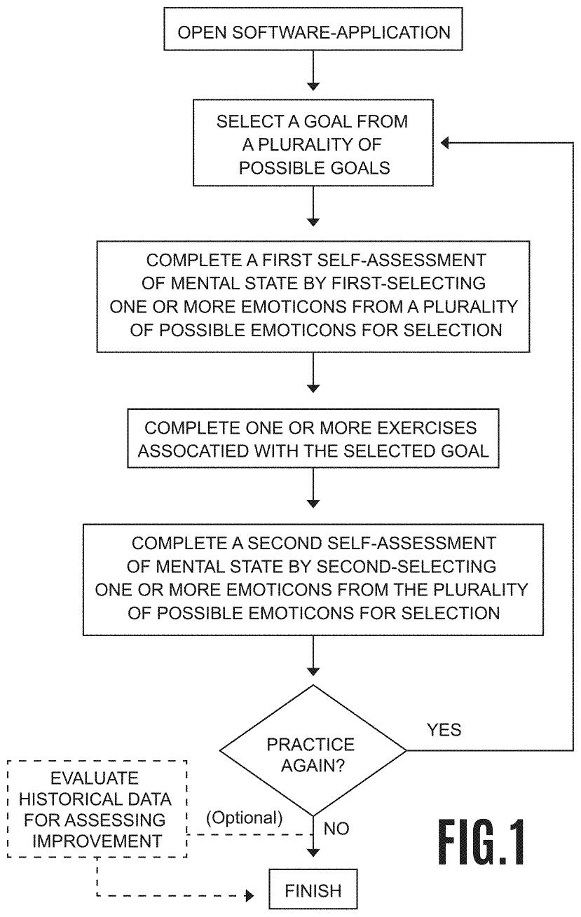

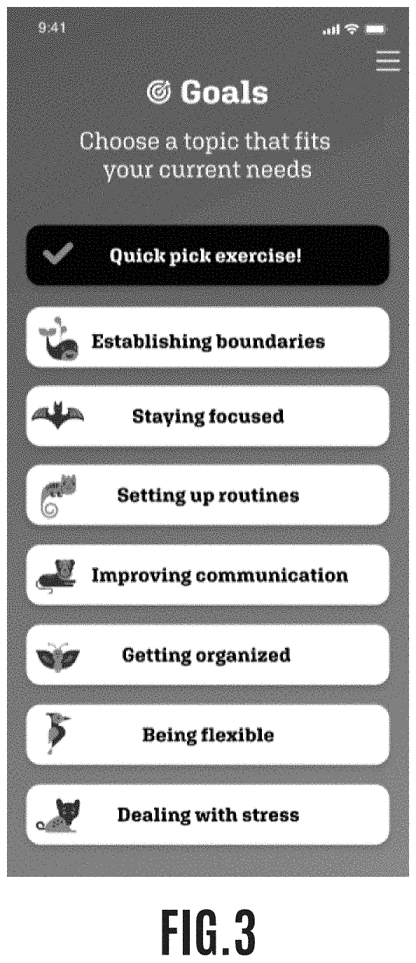

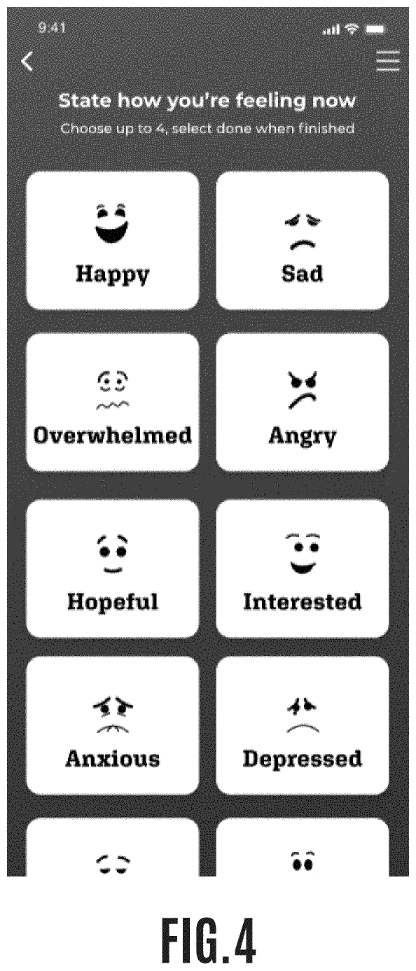

FIG. 1 shows a schematic representing a method for improving cognitive state in a user, such as decision-making, including several method steps for achieving the same. Here, a user opens the software-application; typically, on a smart phone, although other devices may be used to access the software-application, either locally stored, or stored on the cloud (internet network). The user selects a goal form a plurality of possible goals, generally by clicking on an icon (See FIG. 3). The user is prompted to perform a self-assessment of mental state by selecting indicia representing how the user feels; for example, one or more icons may be selected (See FIG. 4), or alternatively, a numerical indicator from a sliding bar or pull down menu, or other indicia may be selected by the user to achieve a substantially similar result. Once the self-assessment is completed, the user is prompted to complete one or more exercises, each of the exercises being associated with the selected goal. In a preferred embodiment, the user is prompted to perform three exercises. After completing each of the exercises, the user is asked to take a second self-assessment, similar to the initial self-assessment. Ideally, the users mental state has improved with the exercises, and results are recorded. The user is prompted with a choice of whether to practice again, in which case the user may select the same or a different goal and repeat the method, or the user may elect to terminate the method for the time being. Optionally, the user may be presented, or able to access, historical data for assessing improvement of mental state or cognitive state.

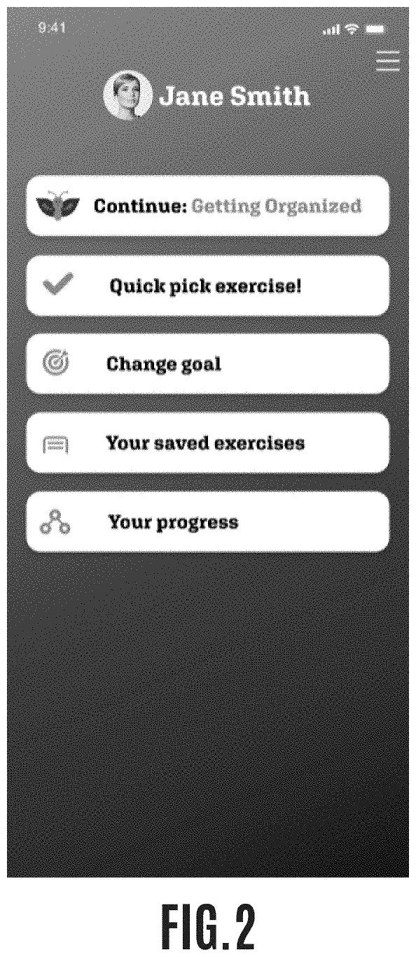

FIG. 2 shows a graphical user interface (GUI), in accordance with one embodiment, for a software-application adapted to execute the method of FIG. 1. Here, the GUI includes a user account indicium, such as name and optional image associated with the user. A menu may be provided for accessing additional functions of the software application, such as a hamburger menu as shown in the upper right corner. In accordance with the illustrated embodiment, one of a plurality of goals can be presented using buttons, although any means for presenting the plurality of possible goals may be provided, such as a pull-down menu, radio selectors, or the like. Here, the user is between goals and able to select (i) an option to continue the “Getting Organized” goal; (ii) a “Quick pick exercise”; (iii) “Change goal”; (iv) “Saved exercises”, and (v) “Your progress”. Selection of one of these options will continue the user to the related activity.

The Quick Pick Exercise may include a random exercise that can be done without having to go through the process of choosing a goal. This daily exercise is derived from one of the plurality of possible goals and is randomly selected to be associated as the ‘quick pick exercise’ for that day.

FIG. 3 shows a GUI for a software-application where one goal can be selected from a plurality of possible goals associated with improving cognitive state of the user. Here, seven goals are illustrated as a plurality of possible goals; however, any number of goals can be similarly provided. The seven goals of the illustrated embodiment include: (i) establishing boundaries; (ii) staying focused; (iii) setting up routines; (iv) improving communication; (v) getting organized; (vi) being flexible; and (vii) dealing with stress. The user selects one of these goals to access related exercises where the user can progress toward improving mental state in association with the selected goal. Optionally, a Quick Pick Exercise, as described above, can be selected in lieu of a goal.

FIG. 4 shows a GUI for a software-application where mental-state indicators are displayed and selectable by the user for performing a self-assessment of mental state. Here, a plurality of icons are presented to the user. The user may select one or a plurality of the icons. In other embodiments, a user might be prompted to enter a numerical value or otherwise indicate how the user is feeling. For clarity, this self-assessment of mental state is preferably administered prior to and after the user engages the exercise(s), in this regard a pre- and post-exercise assessment of mental state can be obtained and monitored for quality purposes.

FIG. 5 shows an example of an exercise associated with a selected goal. Here, the exercise is provided with a descriptive title and two to three sentences describing the exercise. Also indicated might be a duration for performing the exercise and/or location where the exercise is performed. In this example of a first exercise, the user is prompted to think about Actors, and more particularly, the people around the user and what actors might portray those people. In addition, the user is prompted to think about what actor would portray that user. This thought-provoking exercise causes the user to amend mental state and is appropriately titled “Actors”.

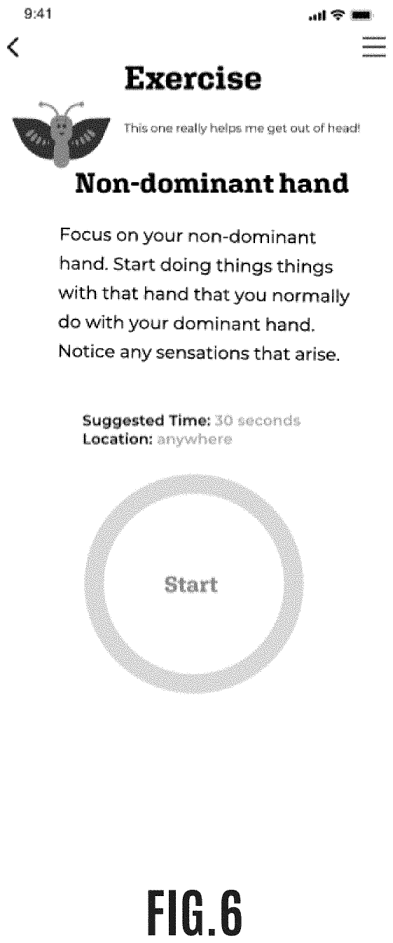

FIG. 6 shows another example of an exercise associated with the selected goal. In this example of a second exercise, the user is prompted to focus on and use a non-dominant hand for common tasks. For a duration of thirty seconds, the user, in this exercise, attempts to use the non-dominant hand in substitution of the dominant hand. In addition, the user is prompted to notice any sensations that arise. This mentally and physically-provoking exercise causes the user to amend mental state and is appropriately titled “Non-dominant hand”.

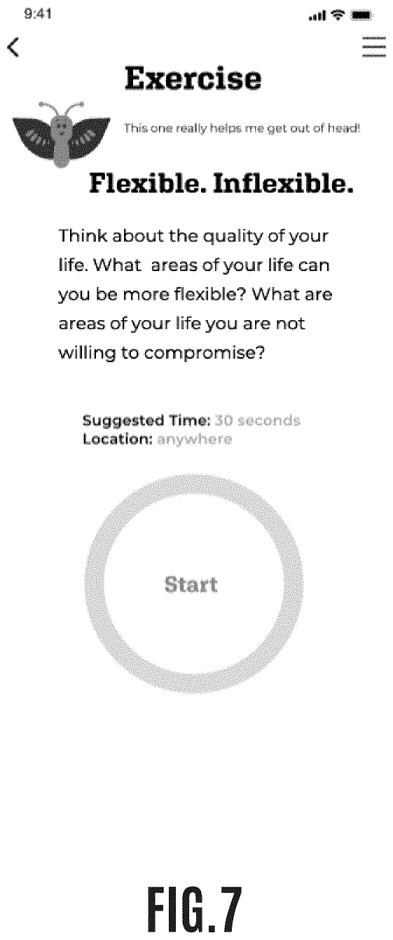

FIG. 7 shows yet another example of an exercise associated with the selected goal. In this example of a third exercise, the user is prompted to focus on quality of life, where the user can be more flexible in terms of quality of life, and where the user is not willing to be flexible or to settle in terms of quality of life. This exercise helps the user find and set boundaries, among other things.

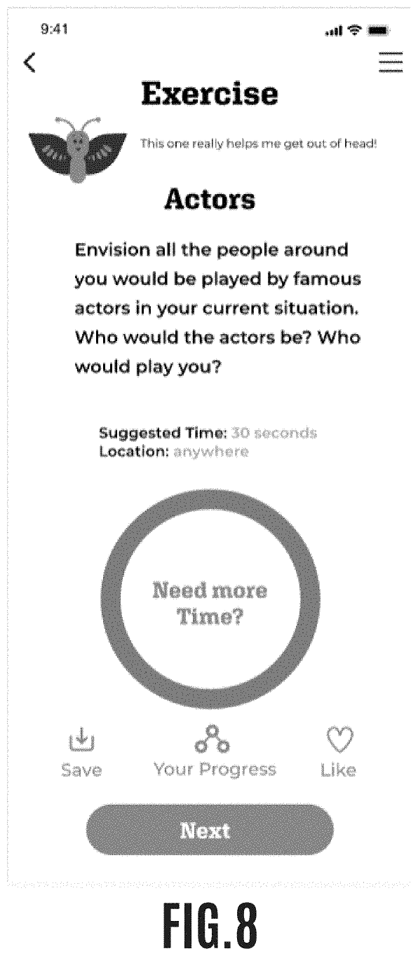

FIG. 8 shows a GUI prompting the user to continue the exercise with more time. Here, the user may be prompted to select a button, for example, a button is highlighted in color after the time duration lapses, to inquire with the user whether additional time is needed to complete the exercise. Additionally, the user may save an exercise and return later for possibly recalling at a later date as one of the user's favorite exercises, view current progress, and/or record a ‘like’ for the exercise to indicate that the exercise worked well for the user.

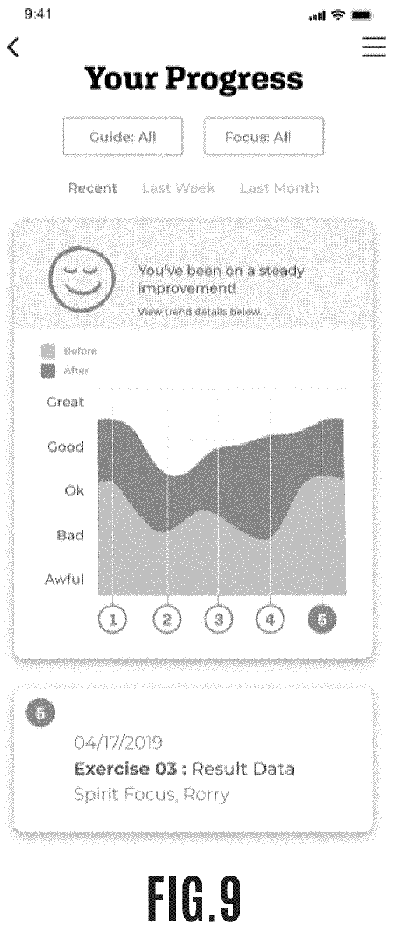

FIG. 9 shows a GUI configured to display historical data for assessing improvement in cognitive state of the user over time. Here, a graphical representation communicates mental state both before and after the exercise(s) performed, and for each completed activity over time. On the dependent axis is generally a quantification of mental state based on the user pre- and post-exercise self-assessments. On the independent axis may be (i) iteration; (ii) date; or other time-related indication. This representation can be used to show relative improvement for each iteration, and/or improvement over time as additional iterations are completed using the software-application.

In accordance with various embodiments, the user may be presented an option to click a link in a reminder notification message for example, from their smart phone, which reminder can prompt the user to complete an exercise. The link may be configured to take the user directly to the Quick Pick Exercise (exercise for the day). Reminder notifications can be set, within the software application, for presentation at every hour, a few times a day or once a day.

Another feature can be provided to allow a user to save an exercise that the user ‘liked’ or that worked well for them, very much like a bookmark or favorite. The user can save up to a maximum number of favorites, for example up to twelve, and can access them from the main menu.

What is claimed is:

1. A method for improving cognitive state in a user, comprising:
executing a software-application configured to present a plurality of exercises to the user on a display screen;
with the software-application:
selecting a goal from a plurality of possible goals, wherein each of said goals is associated with a decision-making activity; and
first-selecting one or more mental-state indicators to obtain an initial self-assessment of the mental state of the user;
followed by:
completing one or more exercises associated with the selected goal; and
second-selecting one or more mental-state indicators to obtain a subsequent self-assessment of the mental state of the user.

2. The method of claim 1, wherein after obtaining the subsequent self-assessment of the mental state of the user, a second goal is selected, wherein the method is repeated according to the second goal.

3. The method of claim 1, wherein after obtaining the subsequent self-assessment of the mental state of the user, the method further comprises: evaluating historical data for assessing cognitive improvement.

4. A system adapted to execute, in connection with a non-tangible computer readable medium, a method for improving cognitive state in a user, wherein the method comprises:
executing a software-application configured to present a plurality of exercises to the user on a display screen;
with the software-application:
selecting a goal from a plurality of possible goals, wherein each of said goals is associated with a decision-making activity; and
first-selecting one or more mental-state indicators to obtain an initial self-assessment of the mental state of the user;
followed by:
completing one or more exercises associated with the selected goal; and
second-selecting one or more mental-state indicators to obtain a subsequent self-assessment of the mental state of the user.

5. The system of claim 4, wherein the display screen is coupled to a mobile phone.

6. The system of claim 4, wherein the display screen is coupled to a personal computer, including: a tablet PC, laptop PC, or desktop PC.

7. The system of claim 4, wherein the non-tangible computer readable medium is stored on an internet network.

8. The system of claim 4, wherein the non-tangible computer readable medium is locally stored on memory associated with a device.
